(19) United States
(12) Patent Application Publication
(10) Pub. No.: US 2022/0007753 A1
(43) Pub. Date: Jan. 13, 2022

(54) TOE PROTECTING APPARATUS

(71) Applicant: Hae Ha KIM, Gyeonggi-do (KR)

(72) Inventor: Hae Ha KIM, Gyeonggi-do (KR)

(21) Appl. No.: 17/295,850

(22) PCT Filed: Nov. 21, 2019
(86) PCT No.: PCT/KR2019/016027

§ 371 Date: May 20, 2021

(30) Foreign Application Priority Data
Nov. 22, 2018 (KR) 10-2018-0145313

(51) Int. Cl. A41D 13/06; A61F 5/30; A41D 13/015; A61F 5/01

(52) U.S. Cl. CPC A41D 13/06; A61F 5/019; A41D 13/015; A61F 5/30

(57) ABSTRACT

A toe protecting apparatus according to the present invention prevents blisters from forming by providing a support part that is thicker on any one side than the other side in the longitudinal direction.

### TECHNICAL FIELD

The present invention relates to a toe protecting apparatus, and to a toe protecting apparatus that prevents blisters from forming on the toes.

### BACKGROUND ART

Prolonged walking and vigorous exercise such as mountain climbing and trekking cause blisters to form on various parts of the feet. For example, new shoes that poorly fit on the underside of the foot cause blisters to form on the soles of the feet, and ill-fitting shoes that are too big or too small cause blisters to form on the heels.

As a patent that prevents blisters from forming on the heels, Korean Patent Publication No. 20150029603 has proposed technologies such as a method of manufacturing size correction pad for only climbing shoes to protect the heels.

Another factor that causes walking discomfort is blisters on the toes. Hereinafter, the blisters on the toes will be referred to as toe blisters. Despite the fact that pedestrians suffer great discomfort due to the toe blisters causing discomfort every time hitting the ground, only a technique of sharing a finger thimble for the toes has been introduced on the Internet as a technique for addressing the issue described above. The toe thimble is nothing more than protecting the toes from fracture.

### DISCLOSURE OF THE INVENTION

### Technical Problem

The present invention, as proposed under the aforementioned background, proposes a toe protecting apparatus capable of preventing blisters from forming on the toes and capable of removing already-existing blisters without pain.

The present invention proposes a toe protecting apparatus that allows comfort in use without irritating an affected toe as well as other toes adjacent to the affected toe.

The present invention proposes a toe protecting apparatus that may be adaptively used in patients and affected areas.

### Technical Solution

The present invention provides a toe protecting apparatus including a base having an opening through which a toe fits to correspond to a branched part of the toe, an end placed on the opposite side of the base, and an encloser, between the base and the end, having a hollow for surrounding the toe and providing a support part which is thicker on any one side than the other side in the longitudinal direction to absorb a torsional force applied to a blister candidate of the toe. According to the present invention, the support part prevents blisters from forming on the toe and reduces pain from existing blisters. The encloser is designed to surround the toe, and thus, there is an advantage in that a separate support member for the toe protecting apparatus is not required.

The support part is formed on the left side of a toe for a toe of the right foot, and is formed on the right side of a toe for a left toe, and may thus be used on the toes of both feet.

The support part protrudes inward from the encloser to increase a thickness of the encloser at a corresponding portion, and may thus prevent other adjacent toes from being affected, and make the adjacent toes less irritated.

The end is designed to be opened to reduce irritation and allow sweat to evaporate.

A wing part extending from an upper side of the base towards the top of the foot is included to prevent the toe protecting apparatus from rotating despite sweating during prolonged walking.

The wing part extends from a gap between toes to prevent the toe protecting apparatus from rotating and reduce feeling of irritation.

An upper portion of the encloser is provided as a straight portion, and a lower portion of the encloser is provided in a curved shape similar to the soft tissue of a toe, and thus providing a similar shape to the toe to allow ease of use.

The encloser is provided with at least two holes to allow sweat to be discharged, so that odor due to sweat deposition in the protecting apparatus may be prevented.

The support part is provided only on an inner surface of the encloser to reduce irritation and pressure applied to other adjacent toes.

A toe protecting apparatus according to another aspect of the present invention includes a base having an opening through which a toe fits to correspond to a branched part of the toe, an end placed on the opposite side of the base, and an encloser, between the base and the end, having a hollow for surrounding the toe and being thicker on a side facing a big toe than a side facing a little toe to absorb a torsional force applied to a blister candidate of the toe, and may thus absorb force applied to toes while walking and absorb force applied to the toes during walking and running.

The encloser may be made of a soft material. For example, silicone may be used.

### Advantageous Effects

According to the present invention, toe blisters that may be caused by lots of walking may be prevented, and already-existing blisters may be quickly removed.

According to the present invention, there is no irritation against other adjacent toes when a toe protecting apparatus is mounted on a blistered toe, and discomfort applied to other adjacent toes may also be avoided.

According to the present invention, only an affected area is adaptively targeted by understanding why lots of walking causes blisters and fixing what is found as a problem, thereby providing no inconvenience in use.

### BRIEF DESCRIPTION OF THE DRAWINGS

FIG. 1 is a schematic view of a toe portion at an end of the right foot;

FIG. 2 is a view showing a direction of force applied to each toe of the right foot when a torsional force is applied;

FIG. 3 is a view showing a portion where the toes are deformed by a torsional force;

FIG. 4 is a view showing a state of deformation of soft toe tissue due to walking;

FIG. 5 is a perspective view of a toe protecting apparatus according to an embodiment;

FIGS. 6 and 7 are cross-sectional views of a toe protecting apparatus and describe main functions of the toe protecting apparatus;

FIG. 8 is a plan view of a toe protecting apparatus having two wings;

FIG. 9 is a perspective view of a toe protecting apparatus according to another embodiment;

FIG. 10 is a right side view of a toe protecting apparatus according to another embodiment; and

FIGS. 11 to 16 are cross-sectional views of a toe protecting apparatus, which are vertically cut through horizontal movement in FIG. 10, FIG. 11 is a cross-sectional view of 1-1′ in FIG. 10, FIG. 12 is a cross-sectional view of 2-2′ in FIG. 10, FIG. 13 is a cross-sectional view of 3-3′ in FIG. 10, FIG. 14 is a cross-sectional view of 4-5′ in FIG. 10, FIG. 15 is a cross-sectional view of 5-5′ in FIG. 10, and FIG. 16 is a cross-sectional view of 6-6′ in FIG. 10.

### MODE FOR CARRYING OUT THE INVENTION

Hereinafter, embodiments of the present invention will be described in detail with reference to the accompanying drawings. The spirit of the present invention is not limited to the following embodiments and those skilled in the art who understand the spirit of the present invention may propose other embodiments included within the scope of the same idea by adding, changing, and deleting components in an easier manner without departing from scope of the spirit of the present invention.

Prior to describing embodiments, results of the inventor's research on the cause of blisters that form on the toes will be described.

First, as a first factor, people walk upright, and thus, a large load is applied to a single foot, and all the loads are concentrated on the toes at a specific point during the action of walking. For example, the toes of any one foot are subjected to the greatest load just before the heel of the other foot reaches the foot.

As a second factor, people's feet are not aligned in a straight line, and their feet are inclined towards the outside at a predetermined angle in many cases. This may have a stable effect for expanding the center of gravity and keeping a standing posture, but causes dynamic characteristics in the toes and acts as a problem of increasing fatigue to the toes.

To be specific, the second factor creates a discrepancy between a direction a pedestrian heads and a direction his/her feet face, and creates the force that the toes are pushed in one direction as a whole.

Specifically, when the first factor acts the most, that is, just before the heel of the other foot reaches the foot, the moment when the maximum force is applied to any one toe (hereinafter referred to as a maximum torsion moment), the force in which the toes are pushed may work the most. At this point, the direction of the force generates a torsional force in which the sole of the foot rotates outwards in the heading direction around the ankle. Accordingly, a clockwise torsion is generated in the right foot when viewed from above, and a counterclockwise torsion is generated in the left foot when viewed from above. Such a phenomenon may typically appear in human body structures.

The moment of maximum torsion creates a repetitive impact on the toes and eventually becomes a factor causing blisters to form on the toes.

FIG. 1 is a schematic view of a toe portion at an end of the right foot.

Meanwhile, viewpoints of all drawings were based on the viewpoint of a person having a toe blister, not the viewpoint of an objective observer. Therefore, in understanding the drawings, a cross section of the toes may be understood as a view observed from a rear side in the heading direction.

Referring to FIG. 1, 1 is the largest toe and 5 is the smallest toe. In addition, a dotted arrow line indicates a heading direction when walking, and a solid line indicates a direction of a torsional force.

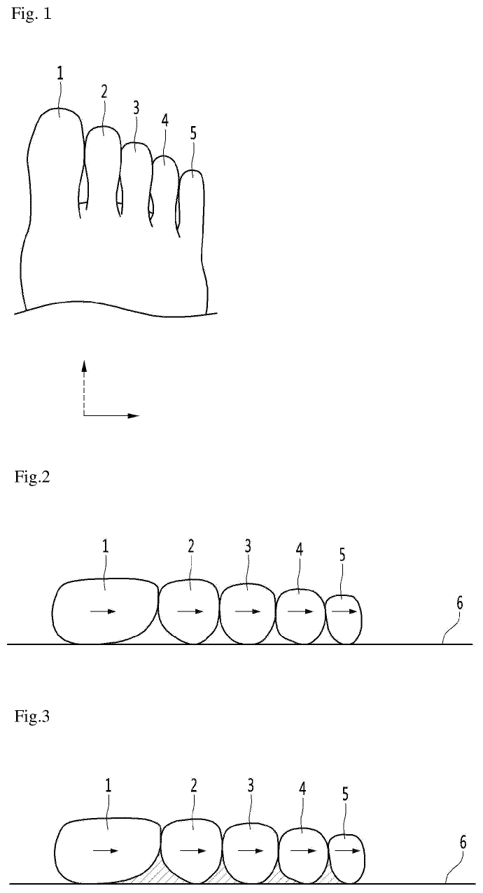

FIG. 2 shows a direction of force applied to each toe of the right foot when the torsional force is applied. Referring to FIG. 2, when a person walks, the torsional force generated by hitting a ground 6 causes the toes to be pushed to the right. In this case, the toe bone is placed in the center of the toes, the soft tissue surrounding the toe bone is inevitably deformed in the end.

FIG. 3 is a view showing a location where the toes are deformed by the torsional force, and referring to FIG. 3, a hatched portion indicates a place where the soft tissue of each toe is deformed and filled when the torsional force is applied. In other words, when the torsional force is applied, the soft tissue is filled at the hatched portion, and without the torsional force, the toes return to their original state.

When such walking motion is repeated, the soft tissue of the toes is eventually deformed. In particular, epidermal cells are released, and thus, fluid leaks and toe blisters form. In this case, the toe blisters mainly form at the most tip of the toes. This is because the load is concentrated in the corresponding portion due to the first factor and the second factor.

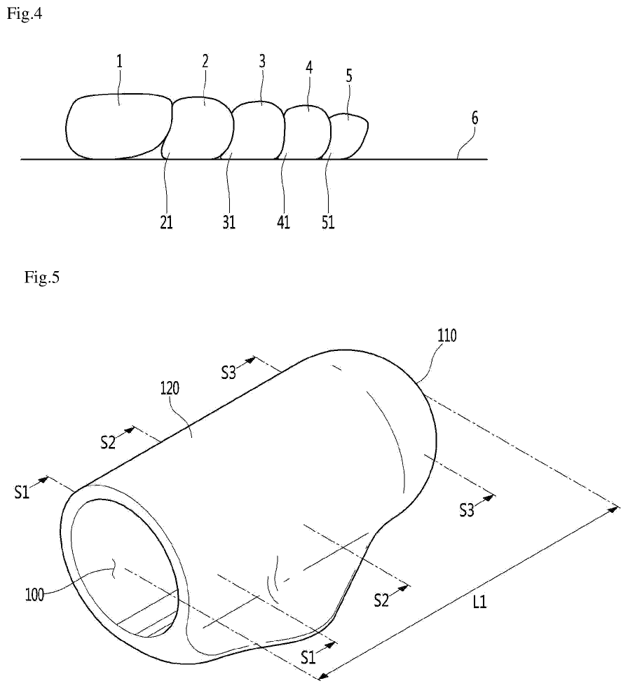

FIG. 4 is a view showing a state in which the soft cell tissue of the toes is deformed due to prolonged walking.

Referring to FIG. 4, it is be seen that repeated load applied to each toe causes the deformation of the soft toe tissues, and accordingly, blister candidates 21, 31, 41, and 51 form. Among the blister candidates, whether to actually develop into a blister depends on a person's walking habit or surgical form, but may be seen mainly in a fourth toe 4 and a third toe 3.

In this case, the blister candidate corresponds to a convex portion located just below the tip of the toes. For example, the blister candidate may be adjacent to a portion where a first joint of the toe tip bone is located. Of course, the exact location of the blister candidate may slightly vary from person to person, but it is seen that the blister candidate is placed right inside the tip. In the present disclosure, the location of the blister candidate is an important point that flows through the content of the specification, and is preferably defined only by the content of the present disclosure.

Once a toe blister forms, the blister causes great discomfort to a pedestrian, which reaches the point where exercise is not doable.

Embodiments are proposed under the above-described background, and a toe protecting apparatus may be an apparatus configured to prevent blisters from forming on the toes or comfortably remove already-existing blisters without pain.

FIG. 5 is a perspective view of a toe protecting apparatus according to an embodiment.

Referring to FIG. 5, the toe protecting apparatus according to an embodiment includes a base having an opening 100 as an entrance end at which a toe starts to be inserted, an end 110 provided on the opposite side of the entrance end, and an encloser 120 surrounding the toe between the base and the end 110. The inside of the encloser is empty so that the toe enters the encloser to face and contact the inside. To this end, materials for the toe protecting apparatus are preferably materials such as soft silicone.

Users may insert his/her toes through the opening 100 and the toes may be placed such that the base reaches hems where the toes are branched from each other. That is, a total length L1 of the toe protecting apparatus may be provided similar to a length of each toe.

The end 110 may be closed or may be open. When the end 110 is open, benefits such as ventilation and reduction of irritation due to toe interference may be obtained. When the end 110 is closed, there is an advantage in that the insertion location of the toe protecting apparatus is more accurately determined and kept as it is even during walking.

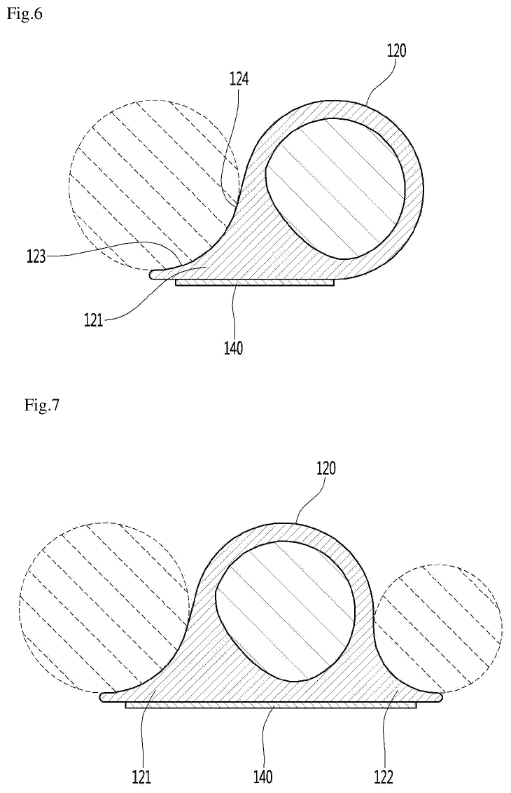

For the shape of the encloser 120 of the toe protecting apparatus, a base end S1 and a tip S3 may be provided the same as the original shape of the toes. On the other hand, a action portion S2 in the approximately middle may be provided with a support part to push blister candidates 21 to 51 inwards. The support part may correspond to a lower left portion of the encloser 120. In FIGS. 6 and 7, the support part may correspond to the lower left portion of the inner space of the encloser, which is hatched by a solid line.

The support part becomes a means of thickening the encloser corresponding to a lower side of the toes to make sure that pushing that causes toe blisters at the location where the toe blisters form does not affect the toes.

Even when the torsional force is applied, the support part serves a role of preventing the blister candidates in the first place, and may apply a weak pressure to already-existing blisters over an entire range to make sure that the already-existing blisters are quickly removed.

For a diameter of the encloser 120 of the toe protecting apparatus, compared to the base end S1 and the tip S3, the action portion S2 may have a largest diameter.

In addition to the support part provided inside the action portion S2, wing parts 121 and 122 are provided outside the action portion S2 to prevent the blister candidate from becoming a blister.

FIG. 6 is a cross-sectional view of an action portion according to any embodiment.

Referring to FIG. 6, between the adjacent toe on a side where the blister candidate is generated and the toe on which the toe protecting apparatus is mounted, the encloser is provided thick in thickness so that the encloser may absorb the torsional force to prevent the blister candidate from becoming a blister. Accordingly, blisters may be prevented from forming on the toes. In this case, the portion where the encloser becomes thick corresponds to a portion where the blister candidate is placed, which may be the lower left and right sides of the encloser.

Inside the encloser 120, the support part is provided in a protruding shape compared to other portions. Outside the support part, one wing part 121 extends outwards. The wing part 121 may be provided on a lower portion of the encloser. In the present embodiment, the wing part 121 may be provided outside the support part. The wing part becomes a means of thickening the encloser to make sure that pushing that causes toe blisters at the location where the toe blisters form does not affect the toes.

The wing part 121 has a shape that becomes thinner towards the outside to make sure that adjacent toes are comfortably placed without irritation. A thick portion between the wing part 121 and the support part may act as a portion that absorbs deformation when the torsional force is applied. Therefore, the blister candidates 21 to 51 may not form on the toes in the first place. A lower surface of the wing part 121 may be provided flat.

In order to further obtain the characteristics described above, an upper portion of the wing part 121 may be provided with a first seating part 124 in which an inflection direction is concave upward in the opposite direction to the other portions of the encloser, and the side portion of the adjacent toe is placed. The thickness between the first seating part 121 and the support part may be thick so that deformation is not applied to the blister candidate of the toes, and blisters may not form. The wing part 121 may be provided thinner towards an edge portion. The lower portion of the wing part 121 may provide a second seating part 123 extending to the lower portion of the adjacent toe. Accordingly, the second seating part 123 is provided in a curved surface similar to the outer shape of the adjacent toe to guide deformation, thereby preventing blisters that may form on the opposite side of the blister candidate of the adjacent toe in advance.

The shape of the wing part 121 corresponds to the adjacent toe, it may thus be possible to prevent the encloser 120 from rotating on the toes.

For reference, a dotted arrow in the drawing indicates an adjacent toe.

Meanwhile, a reinforcement member 140 is for preventing the encloser from being twisted or rotating due to a material of the toe protecting apparatus made of a soft material. The reinforcement member 140 may achieve reinforcing the wing part to which the torsional force is applied by fastening a relatively hard plastic plate to a portion, or by increasing the thickness of the soft member itself providing the encloser.

FIG. 7 is a cross-sectional view of an action portion according to another embodiment.

Referring to FIG. 7, it is different in that the wing part is also provided on the opposite side where the support part is provided. According to the present embodiment, the other wing part 122 absorbs the torsional force applied to the blister candidate of the toes corresponding to the other wing part 122 itself, it may thus be possible to prevent blisters from forming on two toes with one toe protecting apparatus.

Therefore, the shape of the two wing parts 121 and 122 are identical as a whole.

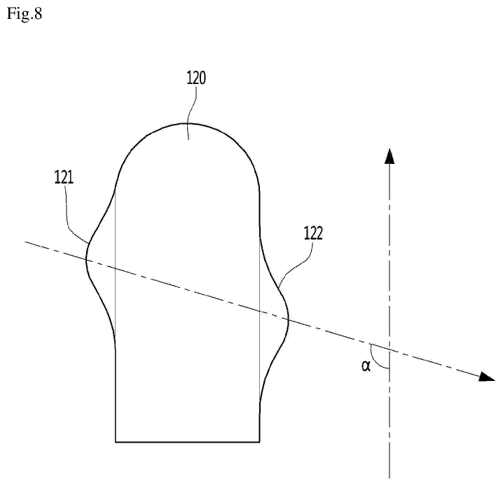

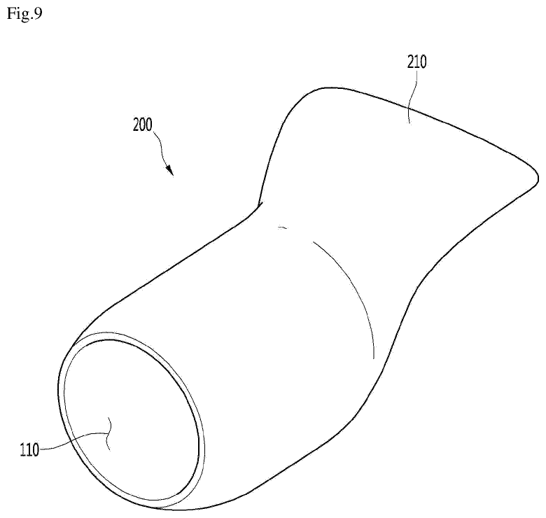

In another embodiment, it is seen that the shapes of the wing parts 121 and 122 are different, because the locations of the wing parts are different from each other. With reference to a plan view of the toe protecting apparatus of FIG. 8, descriptions will be provided in more detail.

Referring to FIG. 8, the direction of the toes (dashed dotted line) and the direction of connecting blister candidates 21 to 51 (solid line) are not perpendicular to each other, but cross each other with an angle alpha. This is because the line for connecting the wing parts 121 and 122 need to be inclined accordingly.

The inventor who disclosed the above embodiments created a different embodiment for the toe protecting apparatus by repeatedly manufacturing and wearing prototypes. Hereinafter, it will be described in detail.

The embodiments of FIGS. 5 to 8 have the following problems.

First, there was no problem for people having thin toes, but people having thick toes have a big problem with irritation in the lower portion of the toes such as wing parts.

Although the wing part may serve a role of preventing the rotation of the toe protecting apparatus, the torsional force is applied to the lower side of the toe protecting apparatus including the wing part, and thus, there was a case where a force to rotate the toe protecting apparatus was applied. This happened a lot when the toes were sweaty and the toe protecting apparatus was tightly fitted to the toes.

In the case of sweating in the toe protecting apparatus, there was a problem in that moisture is deposited on a member constituting a material of the toe protecting apparatus to smell.

It was confirmed that the soft tissue of the toes was actually already deformed a lot to form a triangular shape. As such, the toes are in close contact with each other to be provided in the triangle shape, indicating that there is no space between the toes. Accordingly, in the toe protecting apparatus, involving a large volume member in the gap between the toes caused inconvenience.

The inventor further studied an embodiment to solve the problem described above. Here's something to note. The above improvement is only useful to people who have thick toes and sweat a lot, and is not applicable to those who have thin toes and no sweat. Therefore, it may need to refer to the point that the original embodiment may also be preferably used.

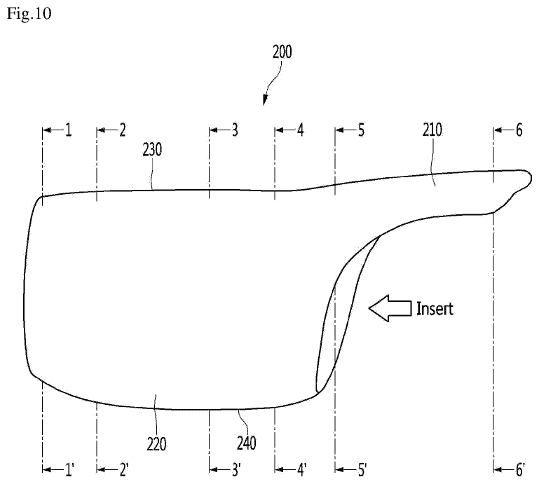

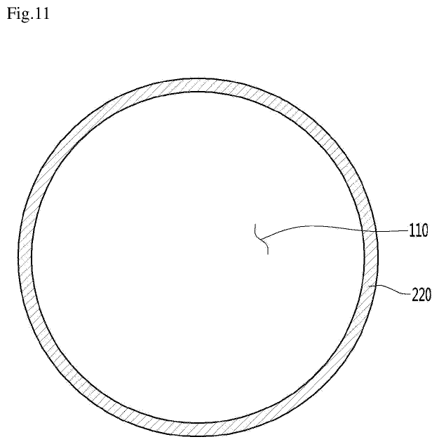

FIG. 9 is a perspective view of a toe protecting apparatus according to another embodiment, and FIG. 10 is a right side view of a toe protecting apparatus according to another embodiment. Embodiments of the drawing may be applied to the toes of the right foot.

Content not specifically mentioned in other embodiments may be applied as it is to other embodiments, except for content that conflicts with the contents of the original embodiment or is not applicable for combination. In the opposite case, similarly, the content of the other embodiment may be applied to the original embodiment except for content that conflicts with the original embodiment or is not applicable for combination.

Referring to FIGS. 9 and 10, a toe protecting apparatus 200 according to another embodiment includes an encloser 220 provided with an end 110 at one end, and a wing part 210 provided on an upper side of an opening 100 at the other end of the encloser 220.

The wing part 210 may extend from an inner end of the toe protecting apparatus to an instep. The wing part 210 extends in contact with the instep which is not sweating often, so that the encloser 220 may be prevented from rotating. In other words, when the torsional force is applied during walking, when the encloser 220 is about to rotate, the left and right ends of the wing part 210 (the upper and lower ends of the wing part in FIG. 10) are caught in a gap between the instep and the toes, thereby preventing the encloser 220 from rotating.

The wing part 210 may be a part where users may hold when the users insert toes into the toe protecting apparatus according to an embodiment. Accordingly, the users may conveniently hold the toe protecting apparatus to wear or remove the apparatus from the toes.

At least two holes are perforated in the encloser 220 and in the wing part 210 to allow sweat from the toes to be discharged.

The upper portion of the encloser 220 is provided as a substantially straight portion 230, and may correspond to the shape of actual toes. Rather than referring to a straight line extending in one direction in a general sense, the straight portion 230 may be understood to include irregularities in a shape corresponding to ups and downs of each toe joint in an engineering and industrial sense.

The lower portion of the encloser 220 may be substantially provided as a curved portion 240. The curved portion 240 may be provided in a shape similar to a convex lower shape of the end joint of actual toes. By the curved portion 240, the toe protecting apparatus keeps a shape corresponding to the shape of the end joint where blisters form on the toes to prevent blisters from forming as much as possible, and to prevent the toe protecting apparatus from rotating due to the torsional force.

In FIG. 10, a place marked ‘insert’ in the arrow indicates an opening and indicates a direction in which the toes are inserted.

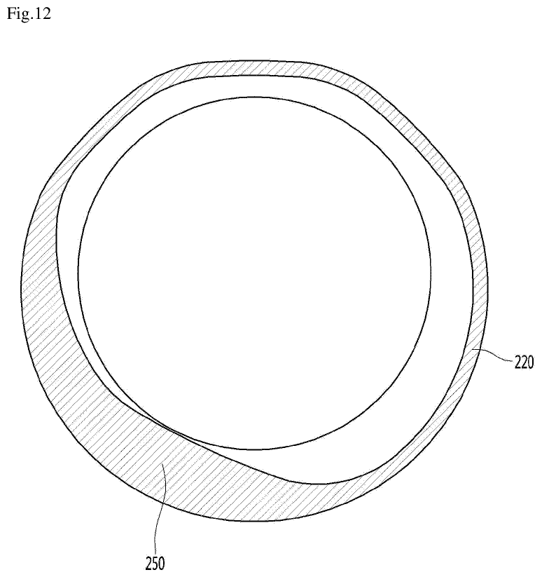

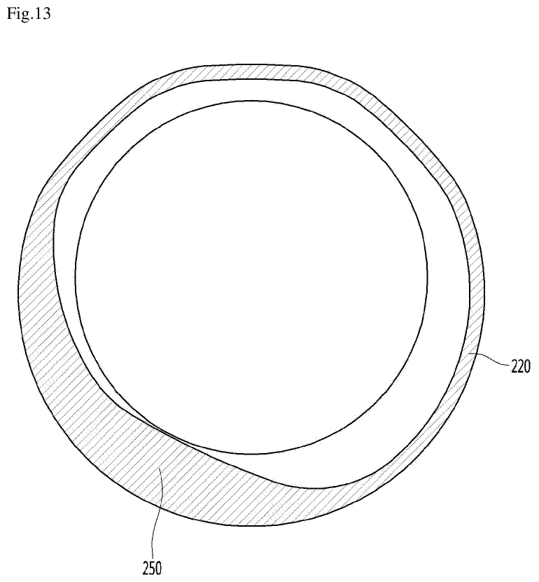

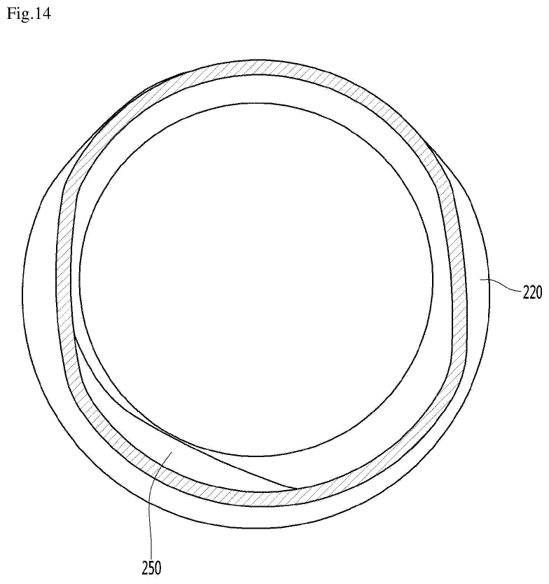

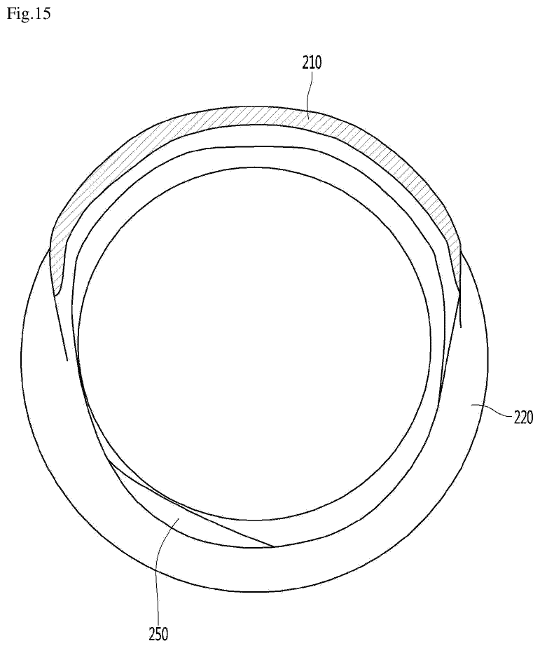

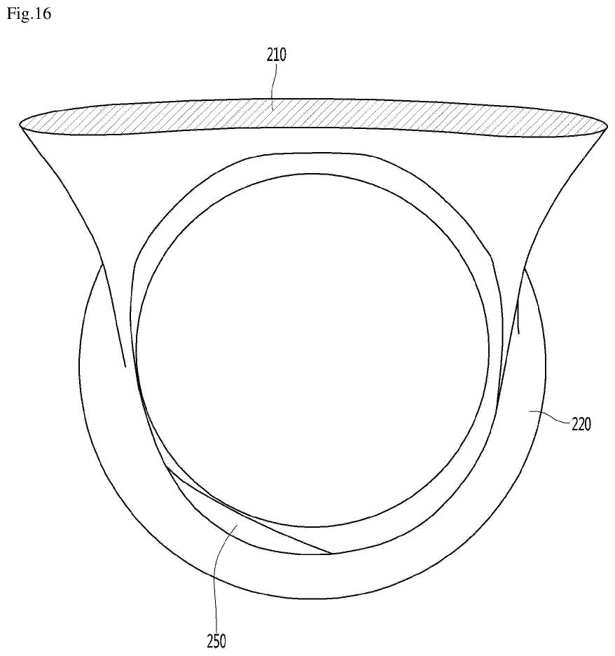

FIGS. 11 to 16 are cross-sectional views of a toe protecting apparatus, which are cut vertically through horizontal movement in FIG. 10. FIG. 11 is a cross-sectional view of 1-1′ in FIG. 10, FIG. 12 is a cross-sectional view of 2-2′ in FIG. 10, FIG. 13 is a cross-sectional view of 3-3′ in FIG. 10, FIG. 14 is a cross-sectional view of 4-5′ in FIG. 10, FIG. 15 is a cross-sectional view of 5-5′ in FIG. 10, and FIG. 16 is a cross-sectional view of 6-6′ in FIG. 10.

Referring to FIG. 11, a portion close to the end 110 may be provided such that the thickness of a wall constituting the encloser 220 is almost the same or equally thin in all directions. For example, at a portion close to the opening 100, the encloser may be provided in the form of a circular frame.

Referring to FIG. 12, at a location where blisters may form due to the torsional force, the thickness of the encloser 220 increases to provide the support part. The support part 250 may absorb strain, which is the deformation of a portion where blisters may form, and prevent the end of the portion where blisters may form from forming a point and protruding.

The support part 250 gradually become thicker, and a portion corresponding to the point of the portion where blisters may form may be the thickest to be provided.

Even when the support part 250 is provided, the support part 250 is formed only on the inner surface of the encloser 220, and the outer surface portion of the encloser 220 is symmetrical with respect to the left and right direction, and may thus be provided in a substantially circular shape.

Referring to FIG. 13, it may be provided in the same shape as that of FIG. 12.

In the middle of FIGS. 12 and 13, the support part 250 may be provided in the thickest shape. Of course, the thickness of the support part may be kept the same in the length direction of the toes. It may be provided differently according to specific aspects of the shape of the toes and blisters that form on the toes. Examples are presented in a shape that the middle part is most convex.

Referring to FIG. 14, except for the location of the support part 250, the encloser 220 may be kept in a shape having the same thickness. However, FIG. 14 has a portion where the shape of the toes extends in a straight line vertically, it may thus be provided in an oval shape vertically extending rather than in a circular shape as shown in FIG. 11.

Referring to FIG. 15, the portion of the encloser 220 surrounding the toes ends, and the wing part 210 further extends from the upper side of the encloser 220 to the rear. The portion where the wing part 210 starts may be provided in a curved shape so as to be surrounded by the upper portion of a root portion of the toes. The wing part 210 may further expand towards the rear. The wing part 210 is sandwiched between the toes, thereby preventing the toe protecting apparatus from rotating.

Referring to FIG. 16, the wing part 210 may be provided in a straight line as a shape corresponding to a flat instep. As described above, the wing part 210 covers at least a portion of the instep, thereby preventing the toe protecting apparatus from rotating.

It was confirmed that blisters did not form when walking was continued by wearing the toe protecting apparatus of all embodiments above.

It was confirmed that when the toe protecting apparatus of all embodiments above is worn in a state where blisters form on the toes, irritation applied to the blisters disappears or becomes weak, having the pain caused by the blisters gone and allowing comfortable walking. However, in this case, it was confirmed that the blisters expanded to the other side of the toes, but the amount of body fluid inside the blisters was the same, and only the two-dimensional range of the blisters was expanded. Even in this case, there was no difficulty in walking because the pain caused by the blisters was gone. In addition, due to the fact that the blisters do not grow, an effect that the blisters were removed quickly was achieved.

### INDUSTRIAL APPLICABILITY

According to the present invention, toe blisters caused by lots of walking may be conveniently prevented. In addition, since a toe protecting apparatus may be manufactured conveniently at low cost, usage thereof holds great promise.

1. A toe protecting apparatus comprising:
a base having an opening through which a toe fits to correspond to a branched part of the toe,
an end placed on the opposite side of the base, and
an encloser, between the base and the end, having a hollow for surrounding the toe and providing a support part which is thicker on any one side than the other side in the longitudinal direction to absorb a torsional force applied to a blister candidate of the toe.

2. The toe protecting apparatus of claim 1, wherein
the support part is formed on the left side of a toe for a toe of the right foot, and is formed on the right side of a toe for a left foot.

3. The toe protecting apparatus of claim 1, wherein
the support part protrudes inward from the encloser to increase a thickness of the encloser at a corresponding portion.

4. The toe protecting apparatus of claim 1, wherein
the end is open.

5. The toe protecting apparatus of claim 1, comprising
a wing part extending from an upper side of the base towards the top of the foot.

6. The toe protecting apparatus of claim 5, wherein
the wing part extends from a gap between toes.

7. The toe protecting apparatus of claim 1, wherein
an upper portion of the encloser is provided as a straight portion, and
a lower portion of the encloser is provided in a curved shape similar to the soft tissue of a toe.

8. The toe protecting apparatus of claim 1, wherein
the encloser is provided with at least two holes to allow sweat to be discharged.

9. The toe protecting apparatus of claim 1, wherein
the support part is provided only on an inner surface of the encloser.

10. A toe protecting apparatus comprising:
a base having an opening through which a toe fits to correspond to a branched part of the toe,
an end placed on the opposite side of the base, and
an encloser, between the base and the end, having a hollow for surrounding the toe and being thicker on a side facing a big toe than a side facing a little toe at lower portion of the encloser to absorb a torsional force applied to a blister candidate of the toe.

11. The toe protecting apparatus of claim 10, wherein
the encloser is made of a soft material.

12. A toe protecting apparatus comprising:
a base having an opening through which a toe fits to correspond to a branched part of the toe,
an end placed on the opposite side of the base, the end being closed, and
an encloser, between the base and the end, having a hollow for surrounding the toe and providing a support part which is thicker on any one side than the other side in the longitudinal direction to absorb a torsional force applied to a blister candidate of the toe, wherein the support part is provided with a lower portion of the encloser.

13. The toe protecting apparatus of claim 12, wherein
the support part is formed on the left side of a toe for a toe of the right foot, and is formed on the right side of a toe for a left foot.

14. The toe protecting apparatus of claim 12, wherein
the support part protrudes inward from the encloser to increase a thickness of the encloser at a corresponding portion.

15. The toe protecting apparatus of claim 12, wherein
below the encloser, a wing part extending to an adjacent toe positioned close to absorb a torsional force applied to the blister candidate is provided.

16. The toe protecting apparatus of claim 15, wherein
the wing part comprises a first seating part whose upper surface is concave upwardly unlike the encloser, such that at least a portion of a side portion of the adjacent toe is placed.

17. The toe protecting apparatus of claim 15, wherein
the wing part comprises a second seating part that extends from the first seating part to a lower portion of the adjacent toe, is thinner towards an edge, and guides an outer shape of the adjacent toe.

18. The toe protecting apparatus of claim 15, wherein
the wing part is provided respectively on both sides of the encloser.

19. The toe protecting apparatus of claim 18, wherein
a line connecting the center of the wing part is not perpendicular to an extension direction of the encloser.

20. The toe protecting apparatus of claim 12, wherein
a reinforcement member for increasing strength is provided on one side of the encloser.
